(19) United States
(12) Patent Application Publication
(10) Pub. No.: US 2015/0187558 A1
(43) Pub. Date: Jul. 2, 2015

(54) PULSE-BURST ASSISTED ELECTROSPRAY IONIZATION MASS SPECTROMETER

(71) Applicant: IMRA AMERICA, INC., Ann Arbor, MI (US)

(72) Inventors: Andrew A. MILLS, Dexter, MI (US); Martin E. FERMANN, Dexter, MI (US); Jiahui PENG, Ann Arbor, MI (US); Robert J. LEVIS, Rose Valley, PA (US)

(73) Assignee: IMRA AMERICA, INC., Ann Arbor, MI (US)

(21) Appl. No.: 14/142,240

(22) Filed: Dec. 27, 2013

(51) Int. Cl. H01J 49/16; G01N 30/96; G01N 33/483; G01N 33/50; G01N 33/68; G01N 33/92; G01N 33/94; G01N 33/02; H01J 49/04; G01N 33/49

(52) U.S. Cl. CPC H01J 49/165; H01J 49/04; H01J 49/0418; G01N 30/96; G01N 33/4833; G01N 33/49; G01N 33/68; G01N 33/92; G01N 33/94; G01N 33/02; G01N 33/5005; G01N 33/5097

(57) ABSTRACT

The present invention relates to a mass spectrometer system, which combines laser desorption with pulse bursts comprising a train of ultrashort pulses and electrospray ionization. The pulse separation between individual pulses within the pulse burst is selected such that transient phenomena on an irradiated sample do not fully relax between individual pulses. Pulses with pulse widths ranging from fs to sub ns are conveniently implemented. The pulse widths can be selected to allow for multi-photon excitation of a sample while at the same time minimizing heat accumulation in a sample. Low cost laser systems such as fiber lasers can be configured to generate appropriate pulse bursts. The technique is suitable for mass spectrometry imaging with high spatial resolution. The laser system can serve as an electronic clock to which the whole mass spectrometry system or mass spectrometry imaging system is synchronized.

### FIELD

The present invention relates to a mass spectrometry system based on pulse-burst assisted laser desorption and electrospray ionization.

### BACKGROUND

A recent innovation in mass spectrometry (MS) is the ability to record mass spectra on samples in their native state, without sample preparation or the necessity of pre-separation with liquid chromatography. A sample is desorbed and ionized at atmospheric pressure via various methods, and generated ions are sent to a mass spectrometer. Rapid analysis is then performed with high sensitivity and high chemical specificity. These characteristics are advantageously applied to high-throughput metabolomics, explosives detection, natural products discovery, pharmaceutical identification, and biological tissue imaging, among other applications. The following exemplary patents, published patent applications, and publications relate to mass spectrometers and in particular pulse laser desorption electrospray ionization mass spectrometry:

Franzen, U.S. Pat. No. 7,193,223, entitled ‘Desorption and ionization of analyte molecules at atmospheric pressure’;

Shiea et al., U.S. Pat. No. 7,687,772, entitled ‘Mass spectrometric imaging method under ambient conditions using electrospray-assisted laser desorption ionization mass spectrometry’;

Shiea, et al., U.S. Pat. No. 7,696,475, entitled ‘Electrospray-assisted laser desorption ionization device, mass spectrometer, and method for mass spectrometry’;

Miller, U.S. Pat. No. 8,029,501, entitled ‘Laser selective cutting by impulsive heat deposition in the IR wavelength range for direct-drive ablation’;

Nemes et al., U.S. Pat. No. 8,067,730, entitled ‘Laser ablation electrospray ionization (LAESI) for atmospheric pressure, In vivo, and imaging mass spectrometry’;

Miller, U.S. Pat. No. 8,110,794, entitled ‘Soft ablative desorption method and system’;

Andrade, et al., U.S. Pat. No. 8,207,494 entitled ‘Laser ablation flowing atmospheric-pressure afterglow for ambient mass spectrometry’;

Levis et al. U.S. patent application Ser. No. 13/390,722, entitled ‘Vaporization device and method for imaging mass spectrometry’;

J. Shiea, et al., ‘Electrospray-assisted laser desorption/ionization mass spectrometry for direct ambient analysis of solids’, Rapid Communications in Mass Spectrometry, vol. 19, pp. 3701-3704 (2005);

M. L. Cowan, et al., ‘Ultrafast memory loss and energy redistribution in the hydrogen bond network of liquid H2O’, Science vol. 434, 199 (2005);

R. G. Cooks, et al., ‘Ambient mass spectrometry’, Science, vol 311, pp. 1566-1570 (2006);

Huang et al, ‘Direct Protein Detection from Biological Media though Electrospray-Assisted Laser Desorption Ionization/Mass Spectrometry’, Journal of Proteome Research, vol. 5, pp. 1107-1116 (2006);

L. Mercadier, et al., ‘Femtosecond laser desorption of ultrathin polymer films from a dielectric surface’, Applied Physics Letters 103, 061107 (2013);

P. Nemes et al., ‘Laser Ablation Electrospray Ionization for Atmospheric Pressure, in Vivo, and Imaging Mass Spectrometry’, Anal. Chem. vol. 79, pp 8098-8106 (2007); and

J. J. Brady, et al., Non-resonant femtosecond laser vaporization of aqueous protein preserves folded structure′, PNAS, vol 108 pp 12217-12222 (2011).

Because mass analysis is performed in vacuum, ionized molecules must be delivered to the gas phase if it is a solid, a liquid, or dissolved in liquid. Solid samples can be ablated from or near the surface.

When a sample is desorbed from a surface and ionized, the produced ions can dissociate into various fragments if the input energy is larger than the molecular ionization potential. Therefore desorption and ionization methods that deliver ionized molecules with a minimal amount of fragmentation are highly sought, these being typically referred to as soft desorption or soft ionization methods. Electro-spray ionization (ESI) is a well-established soft ionization method as for example described in Cooks et al. Soft desorption can further be combined with ESI via the use of appropriate desorption lasers, as for example described in the above '772, '745, '730 and '722 patents. To facilitate laser desorption, resonant absorption via so-called matrix molecules has been implemented. One such example is the resonant desorption performed with lasers operating near the water absorption peak of 3000 nm as discussed in '794 and '730. Alternatively, resonant desorption can also be performed directly taking advantage of typical analyte absorption bands in the UV, as for example described in '772 and '475. In this case, UV pulses with a pulse width of a few nanoseconds (ns) are used. Ns pulse induced desorption is often characterized as a thermal process. In addition to resonant desorption methods, non-resonant desorption methods may be implemented; for example through the use of femtosecond (fs) pulses as described in '722. The desorption process in this case may be induced via multi-photon absorption and may be used even without the use of solvents (sometimes referred to as matrices) and be applied for in-situ analysis of biological or pharmaceutical samples. In this case, the desorption process may occur with minimal heat transfer to the surrounding medium and the desorption process is oftentimes characterized as a non-thermal process.

For non-resonant laser desorption ESI to become a broadly used method in MS, a system architecture that is compatible with low cost desorption lasers would be very beneficial.

### SUMMARY

Benefits of laser desorption ESI via the use of short pulse-bursts are demonstrated. Pulse-bursts enable a particularly soft desorption method as they combine the benefits of non-resonant desorption while minimizing the pulse energy requirements. By distributing the required desorption laser fluence among several pulses in a pulse burst, the amount of molecular excitation and perturbation induced by individual pulses may be reduced. At the same time, the amount of molecular excitation and heat accumulation is gently increased by each individual pulse in the pulse burst, eventually leading to the release of molecules with a minimal amount of excess energy and a minimum amount of fragmentation. The benefits of non-resonant desorption are further preserved by the use of sub-ns (or generally ultrashort) pulses in the desorption process, which can have sufficiently high peak powers to induce multi-photon absorption.

An increase in pulse duration from the fs to ps regime can lower the desorption threshold while extending the interaction time and limiting the amount of heat deposition into the surrounding area, further limiting fragmentation of molecules.

A further benefit of the method of laser desorption ESI with short pulse-bursts is that low-cost laser systems can be used for the desorption process, as the peak power or pulse energy requirements for individual pulses are reduced. Such pulse-bursts can for example be generated with low cost fiber lasers. However, other laser architectures such as solid-state lasers may also be implemented.

Because of the sensitivity at the molecular level to laser induced damage it was not clear undesirable damage could be avoided with multiple pulses. Particularly, as well known in the state of the art, heat accumulation in organic materials may lead to damage. Therefore, it was assumed in the prior art that low repetition rates should be utilized. For example, in U.S. Pat. No. 8,029,501 ‘Laser selective cutting by impulsive heat deposition in the IR wavelength range for direct-drive ablation’ to Miller et al. it was estimated that the time T between individual ablating pulses should be T>(2R)²/6D to avoid damaging heat accumulation, where R is the laser spot diameter and D the diffusivity of the target material. With a value of D˜10⁻⁷/m² for typical organic materials with a high water content and 2R=50 μm, we obtain T 4 ms or a permissible repetition rate of only 25 Hz.

However, results obtained here showed that pulse-bursts provide for a particularly soft desorption method, and allow for laser desorption with molecules intact and without fragmentation. It was determined that a pulse energy slightly above or even below the ablation threshold for individual pulses was acceptable, as will be further discussed below.

The present laser desorption ESI method is compatible with MS imaging, where the use of ultrashort pulses allows for high spatial resolution. To enable MS imaging, the analyte can be positioned on mechanical positioning stages which move the analyte with respect to the focus of the laser beam. Appropriate focusing optics may be also used. Electronics to synchronize pulsing the mass spectrometer to the laser can be used, further ensuring that an equal number of pulses impinges on each measurement spot.

The present laser desorption ESI method is compatible with biologically relevant macromolecules such as: proteins, peptides, lipids, carbohydrates, nucleic acids, DNA, RNA, pathogens, blood, biopsy samples, biological tissue, cells, viruses, animal tissue or plant tissue, serum; as well as: chemical warfare agents, organic molecules, polymers, man-made synthesized compounds, natural compounds, food samples, pharmaceuticals, narcotics, biological fluids, explosives, dyes, nanomaterials or nano-particles.

The present laser desorption ESI method is also compatible with high-repetition rate lasers and is also suitable for implementation with liquid chromatography (LC).

In one aspect the present invention features an apparatus for analyzing samples. The apparatus includes an ultrashort pulse laser generating pulse bursts configured to desorb molecules from a sample in a sample area under ambient conditions. An electrospray ionization (ESI) device is positioned proximate to the sample area. The ESI device is configured to ionize the vaporized molecules under ambient conditions to form ions. A mass spectrometer is included for analysis of the ionized molecules.

A pulse burst may include a pulse train with a repetition rate selected such that transient effects related to the sample do not fully relax between subsequent pulses.

A pulse burst may include pulses with a repetition rate >10 kHz.

A pulse burst may include a train of pulses generated at a first repetition rate, and characterized as being within an envelope of pulses, the envelope generated at a second repetition rate, the second repetition rate being lower than the first repetition rate, wherein the first repetition rate is a pulse repetition rate and the second repetition rate is a burst repetition rate.

A pulse burst may be generated with an optical modulator located upstream of the output from an ultrashort pulse source.

An ultrashort pulse may have a pulse width in the range from about 10 fs up to about 1000 ps.

An ultrashort pulse burst may be generated with a laser source which includes a fiber laser system.

An ultrashort pulse burst may be generated with a laser source which includes a solid-state laser system.

An ultrashort pulse burst may be generated with a laser source which includes a combination of any of a fiber laser system, a solid-state laser, or a diode laser.

A laser source may have an emission wavelength near the 800, 1050, 1550 or 2000 nm wavelength region.

The apparatus may include an optical focusing arrangement, the focusing arrangement configured to focus individual pulses onto the sample with a fluence within a factor of ten of the ablation threshold related to individual pulses.

The apparatus may include an optical focusing arrangement, the focusing arrangement configured to focus individual pulses onto the sample with a fluence within a factor of three of the ablation threshold related to individual pulses.

The apparatus may include an optical focusing arrangement, the focusing arrangement configured to focus individual pulses onto the sample with a fluence lower than the ablation threshold related to individual pulses.

The apparatus may include an optical focusing arrangement, the focusing arrangement configured to focus individual pulses onto the sample with a peak intensity in the range from 0.5 to 5×10¹² W/cm².

The apparatus may include a mass spectrometer.

An ultrashort pulse laser system may be configured to provide a self-referenced precise timing source for the entire mass spectrometry system.

The apparatus may be configured for mass spectrometric imaging.

The apparatus may be configured for cancer detection of tissue retrieved via biopsy.

The apparatus may be configured for in vivo analysis of biological tissue.

The apparatus may be configured to record the mass spectrum of proteins, peptides, lipids, carbohydrates, nucleic acids, chemical warfare agents, DNA, RNA, pathogens, serums, polymers, man-made synthesized compounds, extracted natural compounds, food samples, pharmaceutical compounds, narcotics, explosives, dyes, cells, viruses, human tissue, animal tissue, plant tissue, biological fluids, blood, biopsy samples, nanomaterials or nanoparticles.

In another aspect the present invention features an apparatus for analyzing samples. The apparatus includes a short pulse laser generating pulses configured to desorb molecules from a sample in a sample area under ambient conditions. A pulse width is in the range from about 1 ps to about 1000 ps. The apparatus includes an electrospray ionization (ESI) device positioned proximate to the sample area. The ESI device is configured to ionize the vaporized molecules under ambient conditions to form ions. A mass spectrometer is included for analysis of the ionized molecules.

The apparatus may be applied for desorption of proteins in a folded state.

A sample may include a biological material having a relaxation time in the range from about 100 μs to about 1 ms. The apparatus may provide at least two pulses generated at a pulse repetition rate in the range from about 1 kHz to at least 100 kHz, with additional pulses generated at a burst repetition rate slower than a pulse repetition rate.

### BRIEF DESCRIPTION OF THE DRAWINGS

FIG. 1 schematically illustrates a prior art ultrashort pulse laser desorption electrospray ionization interface for ion mass spectrometers.

FIG. 2 schematically illustrates conventional ultrashort pulse laser desorption and ultrashort pulse-burst desorption.

FIG. 3a is an exemplary embodiment of a focusing arrangement for the laser desorption beam illustrating its near diffraction limited Gaussian shaped mode. Super-Gaussian (or top-hat) spatial modes may also be used in various embodiments.

FIG. 3b is an exemplary embodiment of an alternative geometry for laser desorption ESI.

FIG. 4 schematically illustrates an alternative embodiment of an ultrashort laser desorption ESI system. In this example the femtosecond laser may be used as a timer for synchronization of the mass spectrometry system to the laser source.

FIGS. 5a and 5b contain mass spectra obtained by the exemplary apparatus of this invention of dried Cytochrome C and lipid (DHCP). The top spectrum (5a) shows Cytochrome C dried on a glass slide. The lower spectrum (5b) is of DHCP (1,2-dihexanoyl-sn-glycero-3-phosphocholine). The parent ion is located on the right side of the spectrum with the molecular weight of the parent ion.

FIGS. 6a and 6b are representative mass spectra of dried and liquid blood, respectively.

FIG. 7 is a representative mass spectrum of thin-sections of mouse brain.

FIG. 8 illustrates the survival yield and differentiated internal energy probabilities for molecules desorbed with pulse-burst and supplied in liquid form via nanospray.

### DETAILED DESCRIPTION

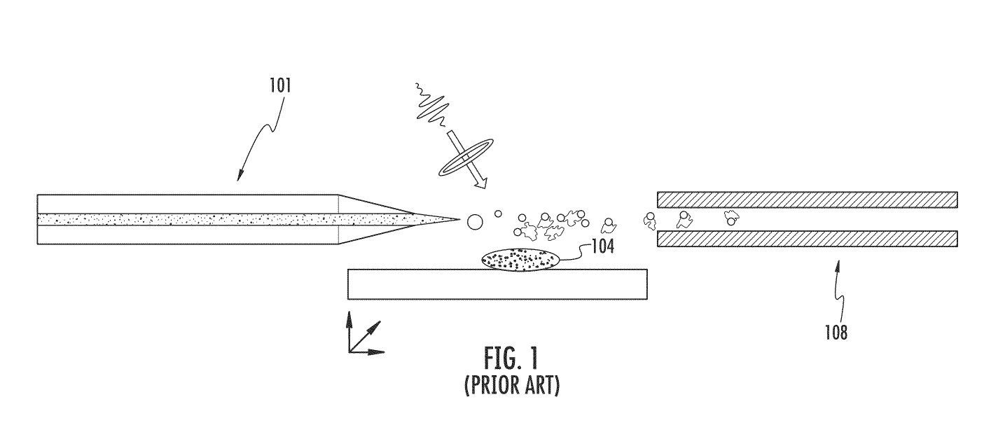

An exemplary prior laser desorption ESI (LESI) ion mass spectrometer may include an interface as shown in FIG. 1. The ESI device operates under ambient conditions (such as temperature and pressure) and adds charge to molecules that are transferred to the gas phase by an appropriate laser desorption beam, shown as incident on a desorption spot. The ESI device is configured to ionize vaporized molecules resulting from laser desorption, under ambient conditions to form ions. The generated ions are collected with a capillary 108 and sent to a mass analyzer. In FIG. 1, the capillary 108 is positioned parallel to a longitudinal axis of an ESI needle 101. This configuration, however, can increase the number of clustered-solvent molecules entering the mass spectrometer system, and therefore many instruments have the spray orthogonal to the capillary axis.

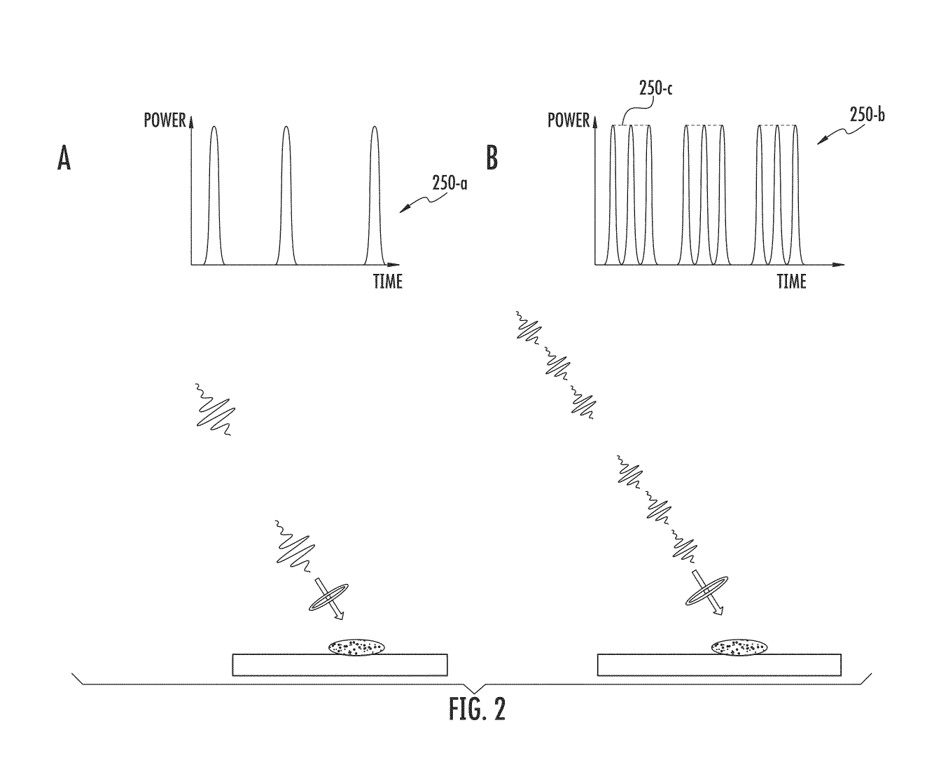

An exemplary illustration of a principle associated with at least one embodiment of the present invention is shown in FIGS. 2a and 2b. Conventional laser desorption ESI (or LEMS) uses a low repetition rate train of pulses 250-a as shown in FIG. 2a for laser desorption. Generally, in a conventional LEMS system the pulse separation at low repetition rates is large enough that the analyte has sufficient time to fully thermally relax between pulses. Typical thermal relaxation times are in the 100 μs-1 ms time frame for biological materials, which corresponds to approximate pulse repetition rates in the 1 kHz-100 kHz range. In certain embodiments of the present invention individual pulses in a pulse burst may be generated at a pulse repetition rate from about 1 kHz up to at least 100 kHz. In various preferred implementations the pulses may be generated more rapidly than the time required for transient effects related to the sample, such as thermal relaxation, to be fully complete. For processing of biological materials, and with consideration of the thermal relaxation times, pulse bursts may readily be generated with a relatively low repetition rate and with various laser sources capable of generating femtosecond or picosecond pulses, for example an ultrashort fiber laser source, or diode pumped solid state ultrashort pulse laser source capable of producing pulse widths in the range from about 10 fs up to about several hundred picoseconds. Pulse-bursts 250-b for various embodiments of the present invention are illustrated in FIG. 2b. Here a train of closely spaced pulses within a burst envelope 250-c (depicted by dashed lines) having a lower burst repetition rate are used. In some implementations a continuous or quasi-continuous train of pulses can also be implemented, however, the pulse separation is chosen so as to not allow the analyte to fully relax between adjacent pulses. This relaxation process can be thermal, but can also comprise any other excitation mechanism related to the analyte, the substrate, a molecular matrix or plume evolution between individual pulses. Such relaxation processes can occur on a time scale in the range of 0.1-100 μs. The use of actual pulse bursts can be of benefit in some biomedical applications, where inter-pulse relaxation phenomena can be exploited, whereas the presence of low repetition rate pulse-burst envelopes allows the minimization of overall heat accumulation.

In some embodiments the laser desorption pulses shown in FIG. 2b are conveniently combined with a conventional laser desorption ESI apparatus as shown in FIG. 1. The ESI component can, for example, be a conventional electro-spray or the newly developed nanospray as well known in the state of the art. Such conventional ESI systems further include an analyzer configured to analyze and detect the generated ions. One advantage of laser desorption ESI is that molecules can be desorbed from a sample or a substrate under ambient conditions. The use of pulse-bursts, among other things, was previously suggested to optimize laser ablation in a different field of endeavor, e.g., micro-machining. This technology is described in, for example, U.S. Pat. No. 7,486,705 ('705): ‘Femtosecond laser processing system with process parameters, controls and feedback’ to L. Shah et al. and U.S. Pat. No. 6,552,301, ('301) ‘Burst-Ultrafast laser Machining Method’, to P. Hermann et al., and U.S. Patent Application Pub. No. 2009/0246530 ('530): “Method of fabricating thin films, to Murakami et al. The '705 patent, '301 patent, and the '530 application are each hereby incorporated by reference in their entirety, However, Applicants are not aware of any prior disclosure regarding the benefits of pulse bursts in desorption of individual molecules.

To facilitate efficient desorption of molecules without or with minimal fragmentation, an ultrashort pulse laser system is used in certain preferred embodiments. One advantage of ultrashort pulse laser desorption is the capability of desorbing large molecules, such as proteins, intact from the condensed phase, as well known in the state of the art. In contrast to conventional ultrashort pulse laser desorption ESI systems, the ultrashort pulse laser system according to this invention is configured to produce pulse-bursts, as described above. The generation and use of bursts using various laser arrangements is known, as exemplified in '705, 301, and '530. As an example, a burst may constitute a first group of pulses generated in rapid succession during an active period and separated from a second group of pulses by a distinguishable inactive period, when viewed on a scale much larger than a minimal pulse separation. The active period may also be characterized by a pulse envelope, the envelope effectively generated at reduced repetition rate compared to the instantaneous repetition rate of pulses in the group. The spacing between pulses in the burst need not be uniform. Likewise, the spacing between bursts (e.g.: envelope spacing) need not be uniform. In accordance with embodiments of the present invention, and from material interaction considerations associated with laser desorption, pulses within a burst are spaced close enough such that the medium does not fully relax during duration of the burst.

With the use of pulse-bursts rather than individual pulses, desorption can be induced with pulses with less peak power. For example, the number of pulses in a pulse-burst can vary from 2-10, 2-100, or 2-2000 and the pulse separation between pulses in a pulse burst can vary between twice the pulse width and up to about 100 μs. As well known in the state of the art thermal relaxation times in organic tissue can be in the range from the μs to the ms range, depending on spot size and penetration depth of the pulses. The pulse width can vary from fs to sub-ns pulses. As discussed above, uniform high repetition rate pulse trains can also produce advantageous effects through the accumulation of molecular perturbations of the analyte, the substrate or other phenomena. In various preferred embodiments the spacing between individual pulses of a burst may be adjustable, as well as the burst repetition rate.

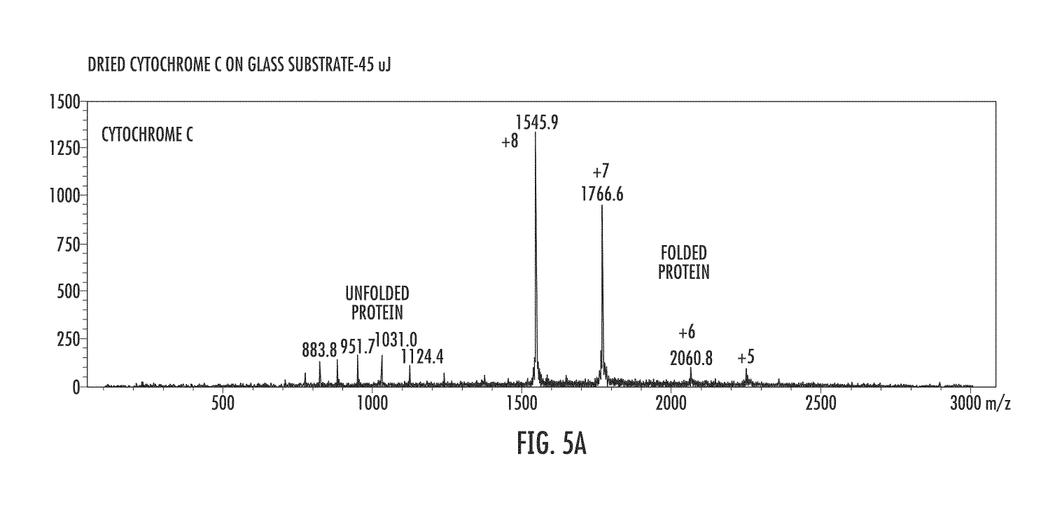

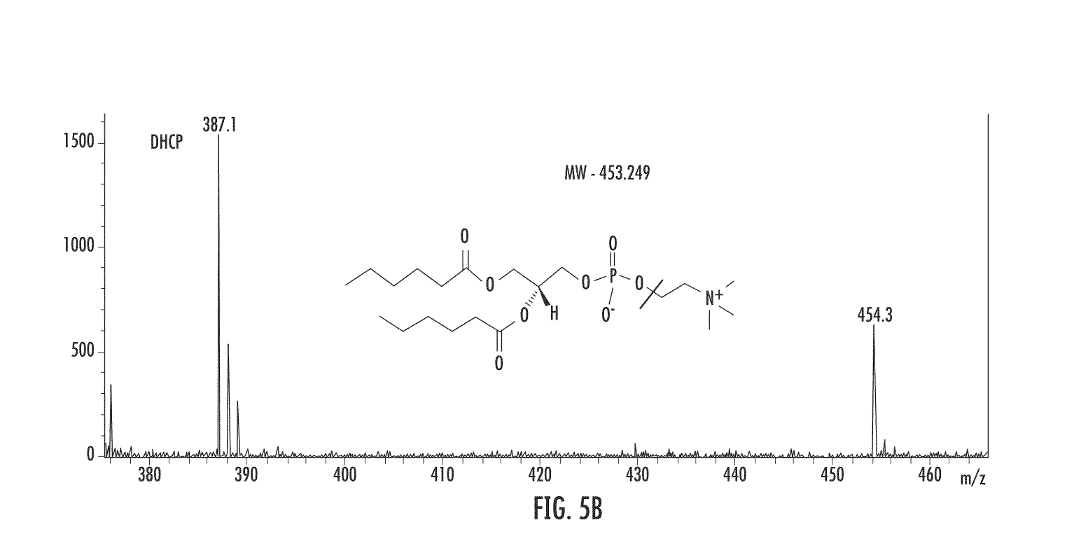

Pulse-bursts can be generated by many different methods. For example, when using a fiber-based chirped pulse amplification system as described with respect to FIG. 5a in U.S. Pat. No. 7,414,780, ('780), ‘All-fiber chirped pulse amplification systems’ to Fermann et al., the pulse picker can be conveniently configured for the generation of pulse bursts in the power amplifier. The '780 patent is hereby incorporated by reference in its entirety. Such systems typically use a mode locked oscillator at the front end which produces pulses at a high repetition rate between 10-100 MHz. The pulse picker, typically configured as an acousto-optic modulator, can then be programmed to select a preferred pulse pattern. For example, a typical pulse pattern could be selected as 111 000 000 000 000 111, where the ones correspond to pulses transmitted by the pulse picker and the zeros are representative of pulses rejected by the pulse picker. For an oscillator repetition rate of 50 MHz, the repetition rate of the burst envelope is thus reduced by a factor of 15, to 3.33 MHz, whereas the pulse separation within the burst is 20 ns and three pulses comprise one burst. Any other suitable pulse pattern can also be selected.

Pulse-bursts can be conveniently generated with fiber amplifier systems comprising oscillators and power amplifiers as disclosed in '780. The use of pulse bursts allows reducing the pulse energy requirements for laser desorption, as the required pulse energy can be distributed among several pulses. This greatly reduces the cost of such laser systems. Moreover the mass spectrometer signal can be improved by simply using several pulses per measurement spot. In addition to fiber laser systems seeded with mode locked lasers, diode laser seeded fiber systems can also be implemented. In fact, with gain-switched diodes in combination with an external pulse generator, optimized pulse patterns for laser desorption can be freely selected within reasonable limitations of pulse energy, rise time, and chirping. Other laser architectures or laser media, such as laser media based on solid-state lasers can also be used to produce pulse bursts. In addition to using electronic means to produce pulse bursts, optical delay lines, pulse shapers, mechanical shutters or beam scanners as well known in the state of the art can also be implemented, alone of in various combinations, as will be further discussed below.

To facilitate laser desorption ESI, the analyte or sample is typically positioned on a substrate for holding the sample, which can be glass, steel, copper, wood, or other materials. However, in-situ, in-vivo desorption of live biological tissue can also be performed, without a substrate. To allow spatially resolved MS imaging, and also to prevent excessive exposure of the analyte to the focused laser beam, the laser beam can be scanned on the sample or tissue in conjunction with a movable ESI needle and mass spectrometer input nozzle. In a simpler imaging configuration, a substrate can be positioned on multi-dimensional translation stages. Such multi-dimensional translation stages or laser beam steering systems are well known in the state of the art and are not further explained here. A particularly efficient imaging configuration is obtained when selecting one pulse burst for each focal spot. This can be accomplished with appropriate synchronization electronics that synchronizes the movement of the translation stages with firing of the laser bursts as will be discussed below.

The limits to spatial resolution are governed by the smallest feature that can be desorbed, the number of molecules in the focal spot as well as the sensitivity of the instrument. The number of molecules desorbed into the gas phase is limited by the amount of energy absorbed in the nonlinear absorption process. Increasing the pulse intensity, or the energy for a fixed pulse duration, can degrade the spatial resolution rather than improving the ablation.

By using pulse-bursts in the desorption process as described here, rather than individual pulses, the desorption process can be improved and the possible spatial resolution optimized. As explained above, the pulse separation within the pulse burst is chosen generally, such that the analyte does not fully relax between individual pulses. The optimum pulse separation depends on the analyte or the molecule that needs to be desorbed and can be optimized with experimentation. For example a pulse separation of 10 μs can be chosen. In MS we have found that pulse-bursts enable a particularly soft desorption method, and allow for laser desorption with a pulse energy slightly above or even below the ablation threshold for individual pulses without fragmentation. For example, the fluence may be within a factor of ten of an ablation threshold related to individual pulses, a factor of three of an ablation threshold related to individual pulses, or somewhat below an ablation threshold related to individual pulses. In various embodiments individual pulses may be focused onto the sample with a peak intensity in the range from 0.5 to 5×10¹² W/cm².

Another way to optimize the desorption efficiency is to keep the laser intensity near the desorption threshold while extending the interaction time by increasing the pulse duration. Preferably, the pulse width increase is selected to still allow for multi-photon absorption while at the same time enabling non-thermal desorption. This can be accomplished with the use of pulses with a width in the range from about 100 fs to about 10 ps, 100 ps, or up to about 1000 ps. In particular, pulse bursts containing pulses with pulse widths in the range from 1-100 ps can sometimes increase the desorption efficiency while enabling non-thermal desorption.

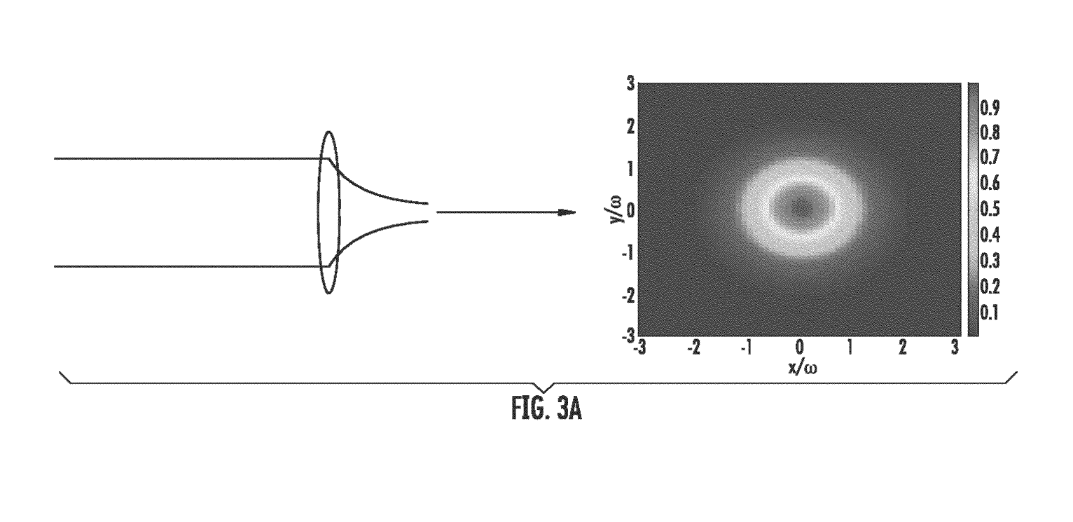

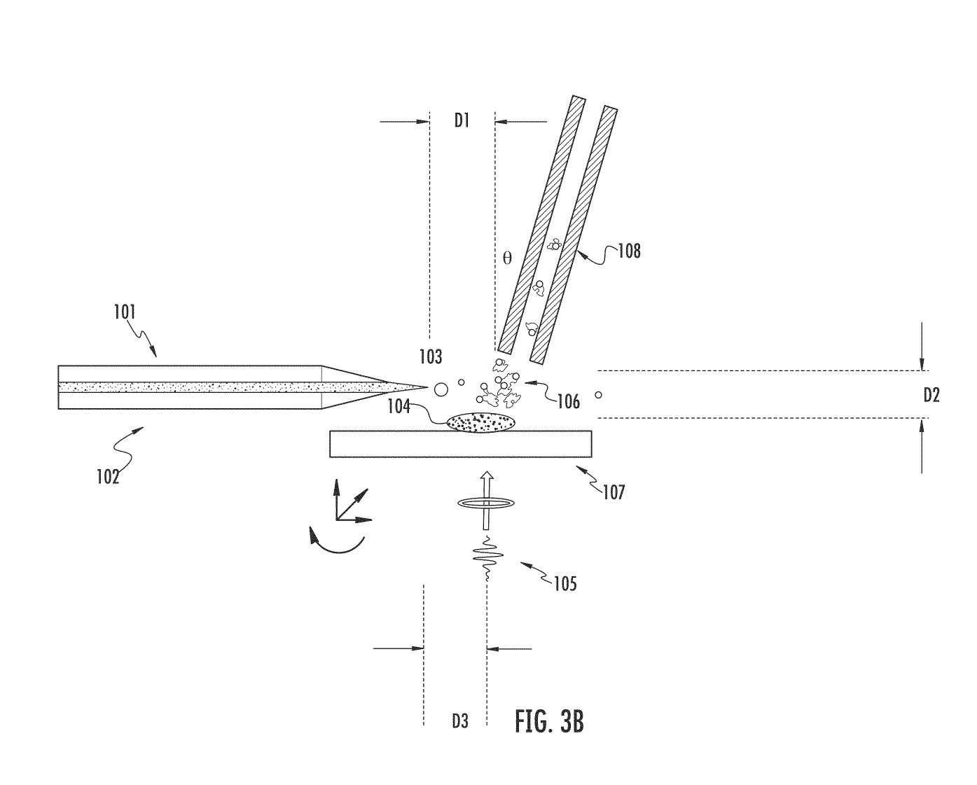

FIG. 3a shows an exemplary focusing arrangement as typically used in conjunction with Gaussian beams for laser desorption ESI, as well as a cross-section of the laser intensity within the beam. It can be seen that the low intensity part of the beam has a much larger focal area than the part with high intensity. In some embodiments it is therefore advantageous to shape the beam in such a way that a top-hat super-Gaussian beam is generated at the focal spot of the laser desorption ESI MS system. This will ensure that the analyte is subjected to the same laser intensity at all points. Methods for producing top-hat beams are well known in the state of the art and are not further explained here.

FIG. 3b is an exemplary embodiment of a laser desorption ESI system using a particular configuration of ESI needle 101, laser beam 105, and mass spectrometer input. In this example the laser impinges from another angle, such as from below the sample. In this case, the ablation plume causes the molecules to travel orthogonally to the ablation surface. In at least one preferred implementation the substrate 107 is located on a multi-dimensional translation stage so that the distance from and angle to the substrate ion collector is optimized with respect to the momentum of the ions. The optical delivery system which includes a focusing lens (not shown) and electrospray needle are also located on multi-dimensional translation stages to optimize the parameters of the ablation plume and ESI spray. One difference between long laser pulse desorption and the ultrashort laser pulse desorption method described here is that the desorbed molecules travel mainly orthogonal to the ablation surface. At the same time, the momentum of the ions has a distribution which is determined by the momentum of the desorbed molecules and the spray droplets. Referring to FIG. 3b, the substrate can be located on a multi-dimensional translation stage so that the distance D1, D2, D3 and angle θ are optimized, where D1 is the distance between the ESI needle 101 and the capillary 108, D2 is the distance between the desorption spot 104 and the capillary 108, D3 is the distance between the ESI needle 101 and the desorption spot 104, and 8 is the angle between the capillary 108 and the vertical line.

In the exemplary setup of FIG. 3b, the substrate 107 is transparent to the laser wavelength being used; it can be a glass or semiconductor, but is not limited to those materials. By way of example, emission wavelengths may be in the UV, visible, near IR or mid-IR wavelengths, for example at or near the 800, 1050, 1550 or 2000 nm wavelength region. Since the desorbed molecules have a large momentum, the final trajectory has a small angle θ. Therefore, the ion collector, capillary 108, can be adjusted to this angle. In this way, the ion collection efficiency is improved, again due to the ultrashort laser matter interaction. Another advantage of this configuration is that the ESI droplets travel at different directions from the ion collector, so that the unwanted ESI background can be removed, an advantageous step for efficient mass spectrometry with the ESI method.

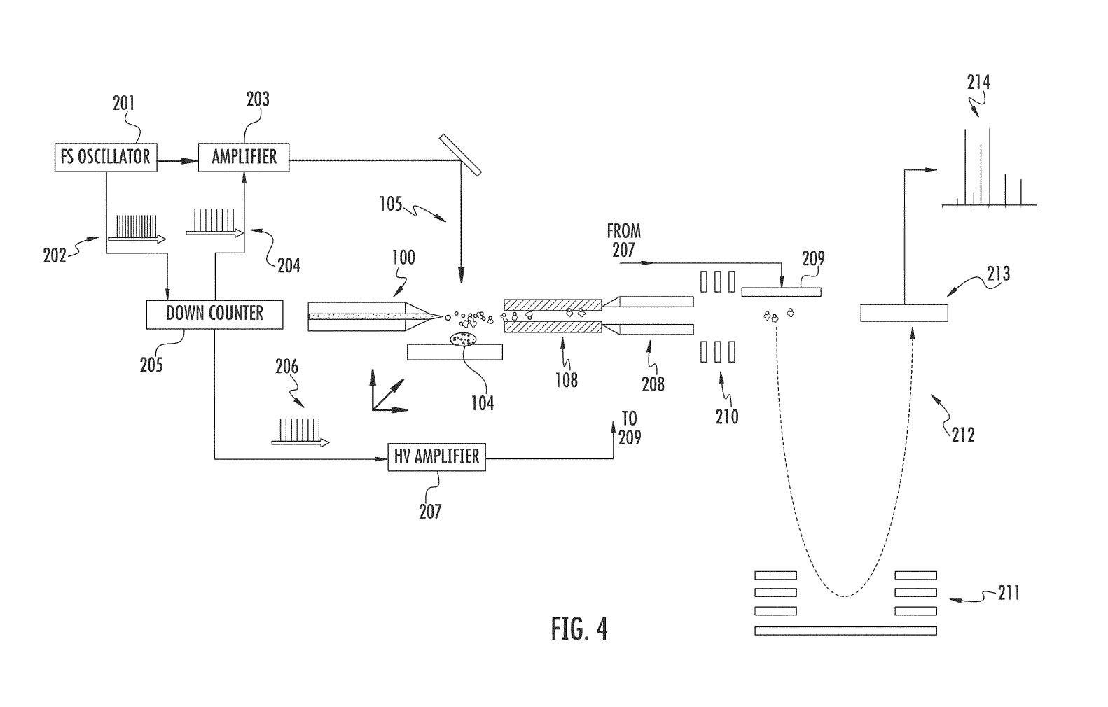

FIG. 4 shows an exemplary mass spectrometer, in a system configuration. A femtosecond laser oscillator 201 generates an ultrashort laser pulse train, with individual pulse-durations ranging from 10 femtoseconds to 1000 picoseconds, or other suitable ranges within the 10 fs-1000 ps range. The pulse trains can generate referenced time signals 202. These signals are received by a down counter 205 to generate trigger signals 204,206 for both an ultrashort pulse laser amplifier 203 and the downstream mass analyzer system which includes high voltage amplifier 207. The system contains an amplified laser beam 105 and an ESI source 100. The generated ions travel through the capillary 108 and pass through a skimmer and quadrupole, hexapole, or some higher-order ion guide 208. After the selected ions traverse the ion guide and ion lens 210, the ion extraction plate 209 sends a high voltage pulse to push the ions into a time-of-flight (TOF) mass analyzer. The ions travel through a trajectory 212 via ion reflector 211 and arrive at the ion detector 213, and generate mass spectrum 214. In FIG. 4, a reflectron TOF mass analyzer is drawn, but the mass analyzer is not limited to such a device, and can be any suitable mass analyzer such as a simple TOF, magnetic sector, RF-Quadrupole, FT-ICR, or Orbitrap. Such MS are well known in the state of the art and not further explained here.

The ion extraction plate 209 can also be triggered via the self-referenced timing signals of the down-counted laser oscillator. In this case, the whole system is synchronized, and the delay between the ultrashort laser desorption and the ion extraction can be optimized for maximum signal. This event-by-event recording makes the system suitable for high spatial resolution and high sensitivity mass spectrometry imaging.

MS timing synchronization not only helps to improve the signal strength, but also helps to improve stability. As discussed above, in order to optimize the desorption process, pulse bursts and/or low intensity ps pulses can be used. Since it is preferable to operate near the ablation threshold, any power fluctuation can cause significant inconsistencies in the desorption process. The use of pulse-bursts is an effective way to minimize fluctuations of desorption efficiency related to pulse instabilities. Given the threshold behavior of ultrashort laser ablation it is beneficial to keep the number of burst-pulses per ablation spot constant; the synchronization scheme described above can be conveniently implemented to ensure that requirement.

To illustrate the benefits of pulse-burst assisted laser desorption ESI, several MS experiments were performed. The system architecture was similar to FIG. 1 with pulse bursts as described in FIG. 2. However, other geometrical arrangements can also be used. The MS system was a commercial Bruker micrOQOF Q-II, equipped with a Nanospray ionization source. We used a commercial ultrashort pulse fiber laser source from IMRA America, Inc. that delivered pulses with a pulse energy between 20-45 μJ at a repetition rate of around 100 kHz. The system includes a fiber oscillator which generates pulses at a high repetition rate, a pulse selector/down counter, and fiber amplifier. The laser wavelength was 1045 nm and the pulse width was around 500 fs. The laser pulses were focused to a 50 μm spot diameter onto the analyte with an angle of around 45 degrees with respect to the substrate normal. A laser fluence of around 0.5-2.5 J/cm² (per pulse) was typically used on the analyte. The peak laser intensity was of the order of 4×10¹² W/cm². The analyte was applied to glass as well as metal substrates. The laser energy was controlled by a wave-plate and polarizer combination. A burst-pulse method was used in such experiments by using two optical choppers, electromechanical devices to further reduce the 100 kHz repetition rate of the commercial laser down to pulse bursts of around 13 pulses at an effective 10 Hz pulse-burst rate. The first chopper consisted of a 10-hole blade with 9 of the holes covered. This chopper was run at an effective frequency of 20 Hz. The second chopper consisted of a 30-hole blade with 29 of the holes covered. This chopper was run at an effective frequency of 130 Hz. The effect of uncovering 10% of the first chopper and 3% of the second chopper yielded a net reduction in the duty cycle passed through each chopper. The 20 Hz chopper had a 5% duty cycle, and the 130 Hz chopper had a 1.6% duty cycle.

An exemplary mass spectrum of dried Cytochrome C and lipid (DHPC 1,2-dihexanoyl-sn-glycero-3-phosphocholine) is shown in FIGS. 5a and 5b respectively, with the sample located on a glass substrate. FIG. 5a shows visible peaks from the right side of the figure at z=5, 6, 7, 8. The higher m/z values distributed in a non-normal distribution indicate folded protein structure, while the Gaussian distribution of lower m/z values with accompanying low intensity indicate that few of the protein molecules are unfolded. FIG. 5b demonstrates that unfolding is completely absent in DHCP. These figures demonstrate that the analyte can be desorbed intact by the ultrashort laser and the exemplary mass spectrometry apparatus is suitable for mass analysis of bio-medical samples.

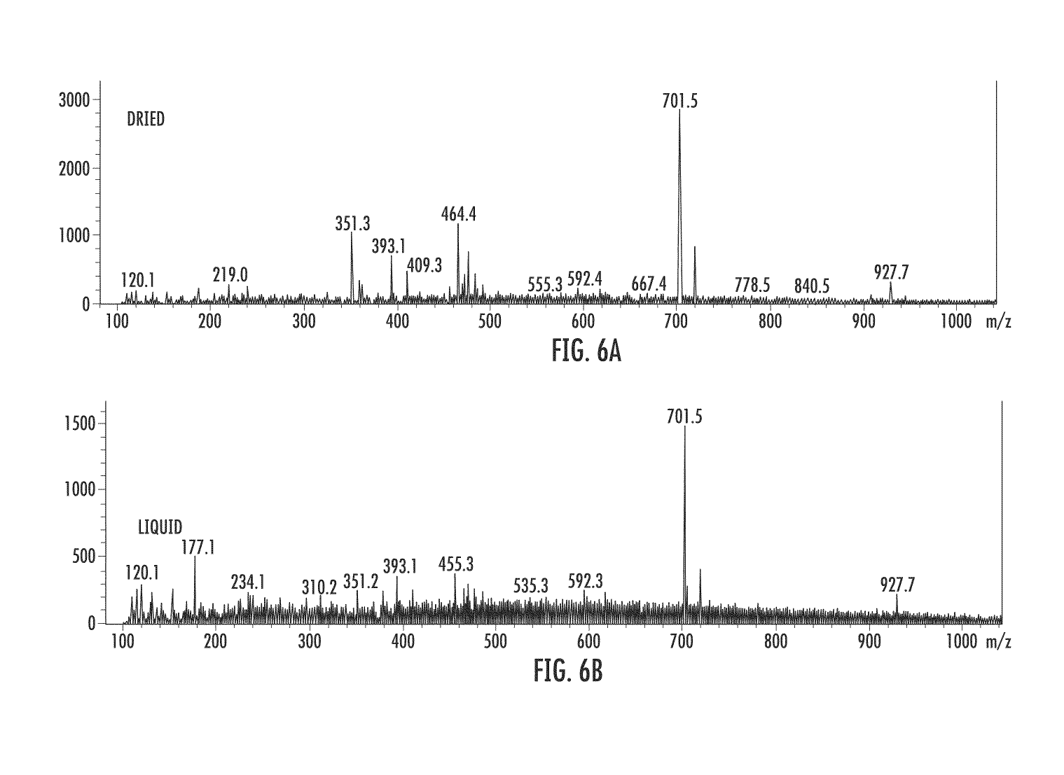

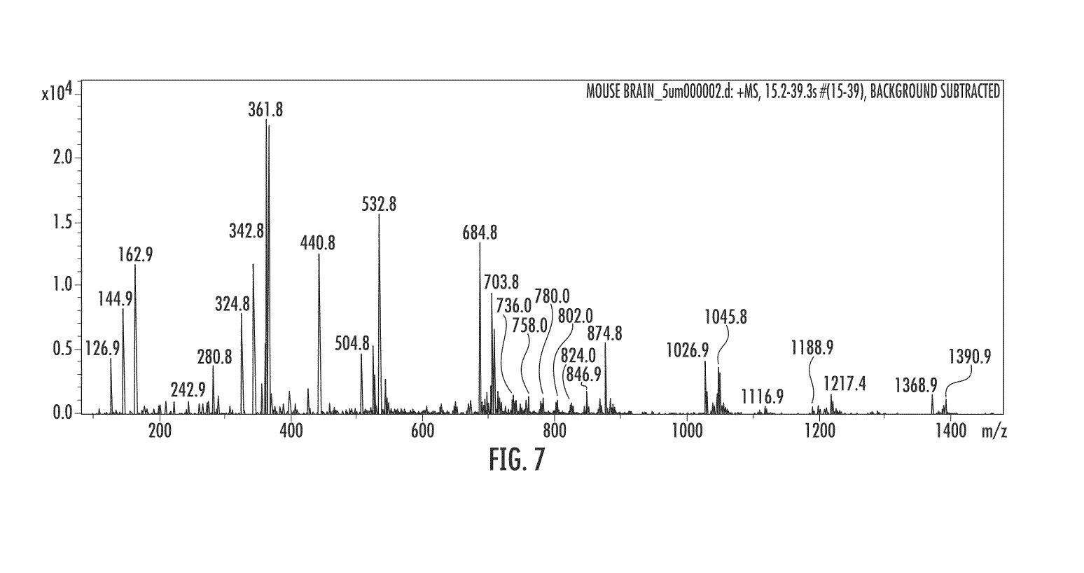

An exemplary mass spectrum of dried and liquid human blood is shown in FIGS. 6a and 6b respectively. While no Hemoglobin peaks are apparent, further investigation is needed to determine what species are being desorbed and ionized. An exemplary mass spectrum of mouse brain is shown in FIG. 7. Several (as of yet) unidentified lipid peaks are apparent. The ability to look at various proteins, carbohydrates, and lipids allows for studying various biomarkers simultaneously without sample preparation. This may enable rapid detection of many diseases, including cancer.

The aforementioned ultrashort laser ablations were conducted in a non-resonant manner. Resonant desorption is also possible. There are some molecules, e.g. H₂O, possessing fast-decaying stretching vibrational states, faster than the heat transfer time. In this case, the energy can be deposited through resonant absorption and cause super-heated molecules to evaporate from the surface. Even in resonant processes, the ultrashort laser duration is beneficial to minimize thermal issues. This ablation method can be used as a way to ablate analyte directly or to ablate analyte indirectly through other solvents or matrices, e.g. H₂O. As in the aforementioned method, the ablated analyte is ionized through ESI.

Fragmentation of ions in a mass spectrometer depends on the internal energy (E_{INT}) of the ions. Unless the molecules are thermalized, they keep a memory of the internal energy received in the source. E_{INT} of the ions can be measured using the survival yield method, which assumes that all ions with internal energy below a critical energy (E₀) do not dissociate (or fragment) and all ions with internal energy above E₀ will fragment. In the method outlined by V. Gabelica et al. (V. Gabelica, ‘Internal Energy and Fragmentation of Ions Produced in Electrospray Sources.’, Mass Spectrom. Rev., 24, 566-587 (2005)), there is a simple fragmentation mechanism of ion fragments, from ions with similar mass, structure, number of degrees of freedom, leading ultimately to similar internal energy distribution. The critical energy is calculated using high-order ab initio methods and has been verified by measurements of survival yield of molecular ions from methods with known internal energy distributions. The survival yield of several molecules with different critical energies (E₀) are measured, plotted, fit to an error function, and differentiated to produce the probability density of ions at various internal energies. The average value is the internal energy of the ions. The survival yield method does not include the kinetic shift (taking into account the amount of time necessary for ions to fall apart), so it can only be used for qualitative measurements, (e.g., comparing Nanospray to pulse-burst desorption ESI post-ionization).

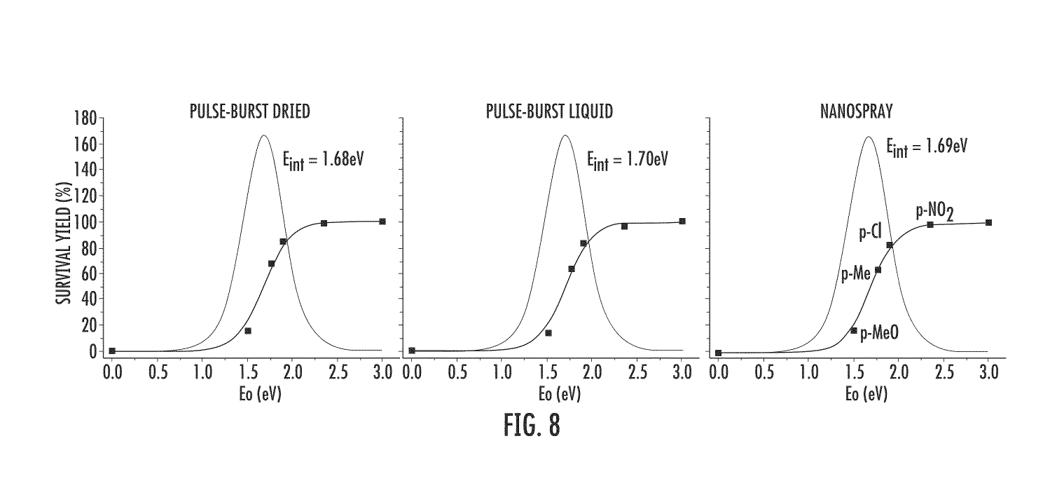

Using the survival method to measure the internal energy distributions of ions entering a mass spectrometer described by V. Gabelica et al., the internal energy of ions ablated with pulse-bursts (post-ionized with ESI) were measured and compared to ions created only by ESI, and found to have no measurable difference in the measured internal energy. Using the pulse-burst method on dried and liquid organic samples followed by ESI post-ionization showed internal energies ranging from 1.68 eV-1.70 eV, as shown in FIG. 8. Samples measured only with ESI showed internal energies of 1.69 eV. This qualitatively illustrates that ultrafast pulse-burst desorption is indeed a soft desorption method complimentary with the soft ionization achieved with ESI.

Other than the applications aforementioned, this invention can also be applied to real time in situ biomedical mass spectrometry analysis. The ultrashort laser desorption serves well as a universal sampling method, and can be easily adapted into different environments.

As illustrated in the above examples, pulse-burst assisted laser desorption is a universally applicable method for biomedical, environmental and pharmaceutical analysis. At the same time, it can improve the spatial resolution and ablation capability and minimize or exploit thermal issues. Ultrashort laser pulses configured as sources of pulse-bursts can also conduct resonant desorption while minimizing thermal issues. Given that the desorbed analytes have better-determined travel trajectories, it can be used to improve the efficiency of ion collection and to remove unwanted ESI background. Burst-pulse techniques help to maintain desorption uniformity, and the synchronization to the laser system. Precise timing helps to maximize the sensitivity of the MS system and is particularly useful in conjunction with mass spectrometric imaging, for both single pulse and pulse-burst operation.

The localization of any material interaction facilitated with the use of ultrashort pulse bursts as described herein makes this invention also suitable for in situ mass spectrometry analysis of biomedical specimens. In particular, the present invention is applicable for medical diagnostics like cancer diagnostics, for example in vivo during surgery, via blood analysis or via the analysis of biological tissue retrieved via biopsy ex vivo. Molecular imaging (2D and 3D) may also be used to examine the biochemistry and metabolism of human tissues, animals and plants.

Although the invention is illustrated and described with reference to some specific conditions, but various modifications may be made in the details for various applications.

While many examples of dried samples have been illustrated, the pulse-burst desorption can also be performed as a high-repetition rate process (i.e.: without choppers). Such a method would be highly suitable for implementation with liquid chromatography (LC).

As used herein, the term “or” is intended to mean an inclusive “or” rather than an exclusive “or”, unless specified. In addition, the articles “a” and “an” as used in this application and the appended claims are to be construed to mean “one or more” or “at least one” unless specified otherwise.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments.

For purposes of summarizing the present disclosure, certain aspects, advantages and novel features are described herein. It is to be understood, however, that not necessarily all such advantages may be achieved in accordance with any particular embodiment. Thus, certain implementations may be embodied or carried out in a manner that achieves one or more advantages without necessarily achieving other advantages as may be taught or suggested herein. No feature or group of features is essential or necessary for each embodiment. Features can be added, removed, or arranged differently than shown or described.

Thus, while only certain embodiments have been specifically described herein, it will be apparent that numerous modifications may be made thereto without departing from the spirit and scope of the invention. Further, acronyms are used merely to enhance the readability of the specification and claims. It should be noted that these acronyms are not intended to lessen the generality of the terms used and they should not be construed to restrict the scope of the claims to the embodiments described therein.

What is claimed is:

1. An apparatus for analyzing samples, comprising:
an ultrashort pulse laser generating pulse bursts configured to desorb molecules from a sample in a sample area under ambient conditions;
an electrospray ionization (ESI) device positioned proximate to the sample area, the ESI device configured to ionize the desorbed molecules under ambient conditions to form ions; and
a mass spectrometer for analysis of the ionized molecules.

2. An apparatus for analyzing samples according to claim 1, each said pulse burst comprising a train of constituent pulses with a repetition rate selected such that transient effects related to the sample do not fully relax between subsequent pulses.

3. An apparatus for analyzing samples according to claim 1, said pulse burst comprising constituent pulses having a repetition rate >10 kHz.

4. An apparatus for analyzing samples according to claim 1, said pulse burst comprising a train of pulses generated at a first repetition rate, and within a pulse envelope, said envelope generated at a second repetition rate, said second repetition rate being lower than said first repetition rate, said first repetition rate being a pulse repetition rate and said second repetition rate being a burst repetition rate.

5. An apparatus for analyzing samples according to claim 1, including an optical modulator located upstream of the output from said ultrashort pulse source, for generating said pulse bursts.

6. An apparatus for analyzing samples according to claim 1, said ultrashort pulses having a pulse width in the range from about 10 fs up to about 1000 ps.

7. An apparatus for analyzing samples according to claim 1, said ultrashort pulse bursts generated with a laser source comprising a fiber laser system.

8. An apparatus for analyzing samples according to claim 1, said ultrashort pulse bursts generated with a laser source comprising a solid-state laser system.

9. An apparatus for analyzing samples according to claim 1, said ultrashort pulse bursts generated with a laser source comprising a combination of any of a fiber laser system, a solid-state laser or a diode laser.

10. An apparatus for analyzing samples according to claim 1, said laser source having an emission wavelength near one of the 800, 1050, 1550 or 2000 nm wavelength regions.

11. An apparatus for analyzing samples according to claim 1, said apparatus further comprising an optical focusing arrangement, said focusing arrangement configured to focus individual pulses onto the sample with a fluence within a factor of ten of the ablation threshold related to individual pulses.

12. An apparatus for analyzing samples according to claim 1, said apparatus further comprising an optical focusing arrangement, said focusing arrangement configured to focus individual pulses onto the sample with a fluence within a factor of three of the ablation threshold related to individual pulses.

13. An apparatus for analyzing samples according to claim 1, said apparatus further comprising an optical focusing arrangement, said focusing arrangement configured to focus individual pulses onto the sample with a fluence lower than the ablation threshold related to individual pulses.

14. An apparatus for analyzing samples according to claim 1, said apparatus further comprising an optical focusing arrangement, said focusing arrangement configured to focus individual pulses onto the sample with a peak intensity in the range from 0.5 to 5×10¹² W/cm².

15. An apparatus for analyzing samples according to claim 1, said apparatus comprising a mass spectrometer.

16. An apparatus for analyzing samples according to claim 15, the ultrashort pulse laser system configured to provide a self-referenced precise timing source for the entire mass spectrometry system.

17. An apparatus for analyzing samples according to claim 15, configured for mass spectrometric imaging.

18. An apparatus for analyzing samples according to claim 15, configured for cancer detection of tissue retrieved via biopsy.

19. An apparatus for analyzing samples according to claim 15, configured for in vivo analysis of biological tissue.

20. An apparatus for analyzing samples according to claim 15, configured to record the mass spectrum of proteins, peptides, lipids, carbohydrates, nucleic acids, chemical warfare agents, DNA, RNA, pathogens, serums, polymers, man-made synthesized compounds, extracted natural compounds, food samples, pharmaceutical compounds, narcotics, explosives, dyes, cells, viruses, human tissue, animal tissue, plant tissue, biological fluids, blood, biopsy samples, nanomaterials or nanoparticles.

21. An apparatus for analyzing samples, comprising:
a short pulse laser generating pulses configured to desorb molecules from a sample in a sample area under ambient conditions,
said pulses having a pulse width in the range from about 1 to ps about 100 ps;
an electrospray ionization (ESI) device positioned proximate to the sample area, the ESI device configured to ionize the desorbed molecules under ambient conditions to form ions; and
a mass spectrometer for analysis of the ionized molecules.

22. An apparatus for analyzing samples according to claim 21, said apparatus applied to the desorption of proteins in a folded state.

23. An apparatus for analyzing samples according to claim 21, wherein said sample comprises a biological material having a relaxation time in the range from about 100 μs to about 1 ms, and at least two pulses are generated at a pulse repetition rate in the range from about 1 kHz to at least 100 kHz, and additional pulses are generated at a burst repetition rate slower than said pulse repetition rate.
